(19) United States
(12) Patent Application Publication
(10) Pub. No.: US 2015/0100016 A1
(43) Pub. Date: Apr. 9, 2015

(54) Manual breast pump

(71) Applicant: Summill International Corp., Taichung (TW)

(72) Inventor: Chi-Min Liao, Taichung (TW)

(73) Assignee: Summill International Corp., Taichung (TW)

(21) Appl. No.: 14/045,802

(22) Filed: Oct. 4, 2013

(51) Int. Cl. A61M 1/06

(52) U.S. Cl. CPC A61M 1/064; A61M 2210/1007
USPC 604/74

(57) ABSTRACT

A manual breast pump is provided with an applicator cup; and a rounded, curved member including a plurality of projections and a coupling releasably secured to the applicator cup. Any two adjacent ones of the projections are separated by a gap. Advantageously, the gaps provide a space to accommodate a flexible deformation of the rounded, curved member when a pumping force is exerted on the breast of a lactating by the pressing.

### BACKGROUND OF THE INVENTION

1. Field of the Invention

The invention relates to breast pumps and more particularly to a manual breast pump with improved characteristics.

2. Description of Related Art

Breast pumps are known device for removing milk from a lactating mother's breasts. For example, a conventional manual breast pump comprising an outer cylinder having a cylindrical wall, an inner cylinder having a cylindrical wall and telescopically disposed in the outer cylinder to form a plunger, the inner cylinder having a closed end and an open end, and a removable cap disposed on the open end of the inner cylinder for closing the open end so that the cylindrical wall of the inner cylinder defines an enclosed interior volume.

Notwithstanding the prior art, the invention is neither taught nor rendered obvious thereby.

### SUMMARY OF THE INVENTION

It is therefore one object of the invention to provide a manual breast pump comprising an applicator cup; and a rounded, curved member including a plurality of projections and a coupling releasably secured to the applicator cup; wherein any two adjacent ones of the projections are separated by a gap.

The above and other objects, features and advantages of the invention will become apparent from the following detailed description taken with the accompanying drawings.

### BRIEF DESCRIPTION OF THE DRAWINGS

FIG. 1 is a perspective view of a manual breast pump according to the invention;

FIG. 2 is another perspective view of the manual breast pump;

FIG. 3 is a side elevation of the manual breast pump;

FIG. 4 is a front view of the manual breast pump; and

FIG. 5 is a rear view of the manual breast pump.

### DETAILED DESCRIPTION OF THE INVENTION

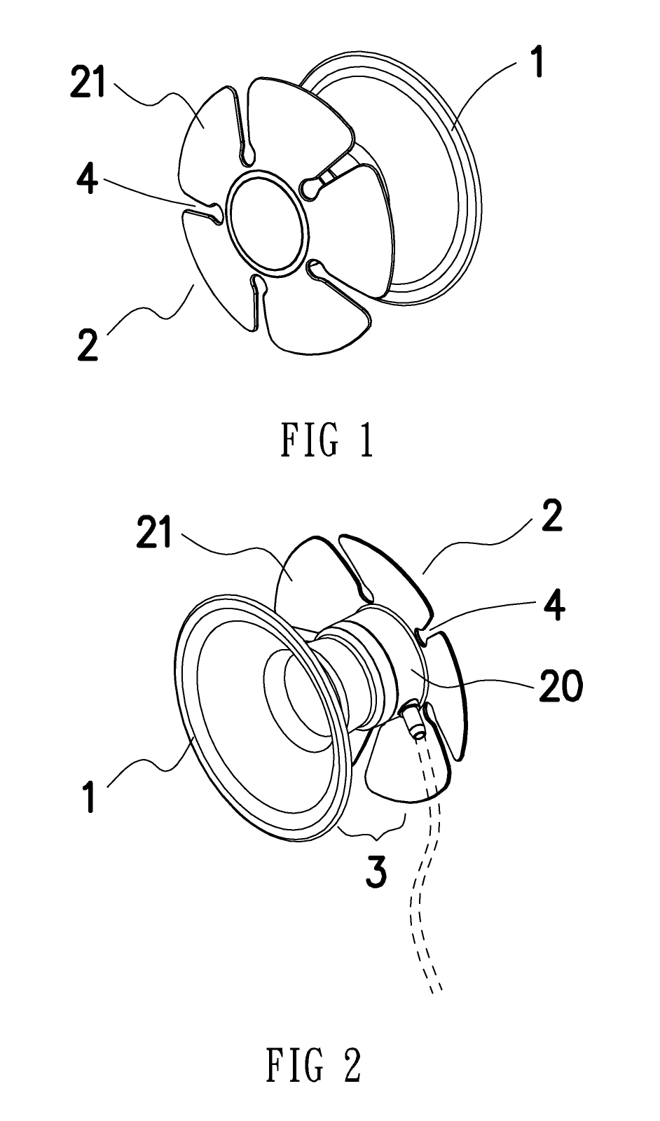

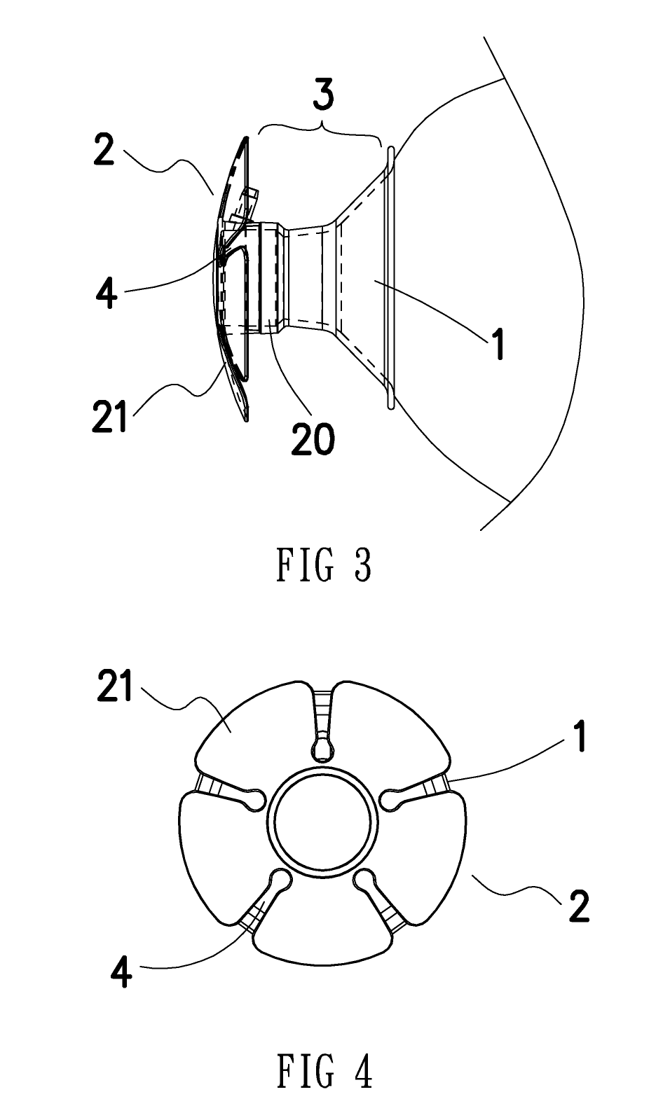

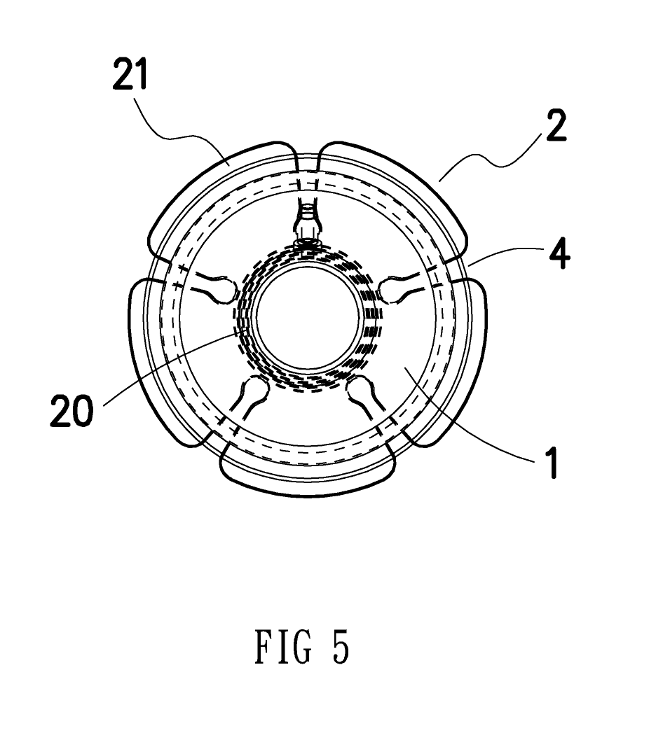

Referring to FIGS. 1 to 5, a manual breast pump in accordance with the invention is made of elastomeric material and comprises the following components as discussed in detail below.

A rear applicator cup 1 is provided. A front rounded, curved member 2 includes five trapezoidal projections 21 and a rear coupling 20 releasably secured to the applicator cup 1. Any two adjacent projections 21 are separated by a gap 4. The rounded, curved member 2 further includes a connector (not numbered) projecting out of the coupling 20 and is in communication with the hollow of the applicator cup 1. A distance between an outer edge of the applicator cup 1 and an outer edge of the rounded, curved member 2 is designated by numeral 3.

In use, a lactating woman may put the applicator cup 1 on her breast. Next, she may press the clothes (e.g., close fitting clothes) put on the breast. Advantageously, the distance 3 and the gaps 4 (mainly) provide a space to accommodate a flexible deformation of the rounded, curved member 2 when a force is exerted on the breast by the pressing. As a result, the woman may feel a degree of comfort while expressing breast milk by a manual pumping operation. It is noted that breast milk extracted from the breast flows to a container (not shown) for storage after passing through the connector and a tube (not shown).

While the invention has been described in terms of preferred embodiments, those skilled in the art will recognize that the invention can be practiced with modifications within the spirit and scope of the appended claims.

What is claimed is:

1. An elastomeric, manual breast pump comprising:
an applicator cup; and
a rounded, curved member including a plurality of projections and a coupling releasably secured to the applicator cup;
wherein any two adjacent ones of the projections are separated by a gap; and
wherein the projections are trapezoidal.
